(19) United States
(12) Patent Application Publication
(10) Pub. No.: US 2007/0232817 A1
(43) Pub. Date: Oct. 4, 2007

(54) Production process of biodiesel from the esterification of free faty acids

(75) Inventors: Antonio Telhado Pereira, Araxa (BR); Kensley Alves de Oliveira, Araxa (BR); Robson de Souza Monteiro, Araxa (BR); Donato Alexandre G. Aranda, Rio de Janeiro (BR); Rafael Thomaz P. Santos, Rio de Janeiro (BR); Rafael Richard Joao, Rio de Janeiro (BR)

Correspondence Address: BIRCH STEWART KOLASCH & BIRCH, PO BOX 747, FALLS CHURCH, VA, 22040-0747, US

(73) Assignee: COMPANHIA BRASILEIRA DE METALURGIA E MINERACAO, Sao Paulo (BR)

(21) Appl. No.: 11/396,637

(22) Filed: Apr. 4, 2006

(51) Int. Cl. C07C 51/43

(52) U.S. Cl. 554/174

(57) ABSTRACT

This invention describes a process for the production of biodiesel from the alcohol esterification of free fatty acids, resulting from the refining of vegetable oils, rejects of industrial and frying oils, and animal fats and organic sewage, using niobic acid as a solid acid catalyst.

### FIELD OF INVENTION

This invention describes a process for the production of biodiesel from the esterification of free fatty acids, resulting from the refining of vegetable oils, rejects of industrial and frying oils, and animal fat and organic sewage, using niobic acid as a solid acid catalyst.

### BACKGROUND OF INVENTION

The transesterification or alcoholysis of vegetable oils is the most used process for the production of biodiesel in industrial scale. In a published work—“Transesterification of Vegetable Oils: a Review” (Journal of Brazilian Chemical Society, vol. 9, no. 1, pp. 199-210, 1998)—Schuchardt and colleagues emphasize that among the different alternatives to improve and use vegetable oils as biofuels, transesterification has been the preferred technology due to the simplicity of the process and the physical-chemical characteristics of the produced biodiesel which is very similar to petroleum diesel, allowing its direct use in Diesel engines with no modifications. The transesterification process of vegetable oils consists in the reaction of the triglycerides (main component of a vegetable oil whose composition is 1 mol of glycerol to 3 mols of fatty acid) with an alcohol in the presence of either acid or basic catalysts. Reaction products are usually a mixture of alkyl esters of fatty acids, glycerol, alcohol, catalyst, and tri-, di- and monoglycerides not completely reacted.

Basic catalysts such as NaOH, KOH, carbonates or alkoxides are industrially more used, as they are more effective than acid catalysts. However, the use of basic catalysts requires the use of refined oils without a significant presence of free fatty acids, otherwise, there would be the occurrence of saponification reactions, reducing the yield of the transesterification. The cost of the refining and the need of cheaper raw materials with the presence of free acidity and impurities has lead to the development of new processes where one step of pre-esterification with acid catalysts is employed, as described on the U.S. Pat. Nos. 4,164,506 and 4,698,186. The disadvantage would be the difficulty to remove the residue of the pre-esterified material catalyst, and then the use of enzymes (U.S. Pat. No. 5,713,965) or barium or calcium acetates (U.S. Pat. No. 5,525,126) as catalysts has been suggested, once it makes the removal of the catalyst easier or eliminates the need of the pre-esterification step, respectively.

The esterification of fatty acids is an alternative for the production of biodiesel (US Patent Application 2004/0254387), as it allows the use of free fatty acids, resulting from the refinement of vegetable oils, animal fats, rejects of industrial and frying oils, and also fatty-enriched organic sewage with significant free acidity. The low aggregated value of these free fatty acids allows the production of biodiesel with extremely competitive prices to petroleum diesel. The U.S. Pat. No. 5,972,057 describes a methodology for the production of biodiesel from the use of rejects of edible oils from restaurants, food industry, home sewage and others. Costa Neto and colleagues (Quimica Nova vol. 23, no. 4, pp. 531-537, 2000) showed that it is possible to produce biodiesel of excellent quality from soy oil used in frying.

Differently of transesterification, the esterification requires the use of an acid catalyst to mediate the reaction of a fatty acid with an alcohol producing fatty acid alkyl ester and water. The acid catalysis may be performed through the use of a homogeneous catalyst such as, for example, sulfuric acid, nitric acid and hydrochloric acid or heterogeneous such as, for example, zeolites and acid resins. The presence of water in the reaction medium considerably decreases the activity and life of the catalyst (U.S. Pat. No. 5,578,090), specially in heterogeneous catalysts, therefore requiring the development of catalysts able to deal with large amounts of water in the reaction medium at the same time that it keeps its activity and stability. The use of heterogeneous catalysts would also provide the benefit of more sustainable processes from the environmental point of view, as it would avoid most of the problems associated to homogeneous catalysts such as corrosion, generation of toxic residues resulting from the excessive use of catalysts, and the difficulty in the separation and recovery of the catalyst.

The hydrated niobium pentoxide (Nb₂O₅.nH₂O), also known as niobic acid, presents a high acidity in its surface and may be used as a solid acid catalyst (Brazilian Patent PI 8306994-1). The acid strength of its sites is equivalent to the acid strength of a 70% sulfuric acid solution when calcined at relatively low temperatures (100 to 300° C.). As it has a significant amount of water in its composition, the niobic acid is able to show higher activity and stability for acid reactions where the water molecules take part as reagents or are released as products compared to other solid acid catalysts. In a review article entitled “Catalytic Application of Niobium Compounds” (Catalysis Today 78, pp. 65-77, 2003), Kozo Tanabe describes the use of niobic acid in the esterification of acetic acid with ethanol and acrylic acid with methanol. In both reactions, the selectivity for the ester product was of 100% with conversions higher than 90%, keeping the performance for longer periods than any other acid catalyst. In a comparative example, an acid resin (Nafion-H) deactivated after an 1-hour reaction, while the niobic acid kept its stable activity and selectivity after a 60-hour reaction, in the esterification of acetic acid with ethanol.

An important difference between acetic acid and fatty acids is that, most of fatty acids have insaturations in their own chains or together with non-saponifiable materials accompanying them such as, for example, esqualene. The presence of insaturations, typically double bounds —C═C—, may promote polymerization, leading to the formation of coke and consequently deactivating the catalyst. As the polymerization is promoted by specific groups of acid sites, it is observed that low SAR zeolites are rapidly active for esterification reactions, but deactivates by the formation of coke, not allowing its commercial use. Other solid acids such as sulfated or tungsten-doped zircons, silica-aluminas and phosphates are also not appropriate as they quickly deactivate.

Another limiting factor for the application of solid acid catalysts in liquid phase reactions where water molecules react or are formed as product is that the water, when adsorbed in the catalytic sites, destroys or notably decreases the activity and lifetime of the catalyst. According to T. Okura in its publication entitled “Water-Tolerant Solid Acid Catalysts” (Chemical Reviews, vol. 102, pp. 3641-3666, 2002) few solid acid catalysts, among them acid zeolites, mixed oxides such as heteropolyacids and phosphates, show acceptable activity, stability and insolubility for liquid phase reactions. This limitation has prevented the development and industrial application of heterogeneous catalysts, and conventional homogeneous catalysts such as sulfuric acid and aluminum chloride are still widely used, in spite of the problems such as catalyst toxicity, corrosion, separation, reuse, and discharge.

This is the case of the esterification of free fatty acids, whose presence of long-chain carboxylic acids (C₆₋C₂₄) makes the reaction to be carried on in liquid phase, and the presence of insaturations in the carbon chain leads to the polymerization processes. The use of an alcohol as esterifying agent produces water in the reaction medium. To perform this reaction using a solid acid catalyst, this catalyst must keep its activity in the presence of water and also there may be no significant polymerization of unsaturated fatty acids, leading to the quick deactivation by the formation of coke, decreasing the lifetime of the catalyst. In addition, the use of a solid acid catalyst would make the product separation process easier allowing its reutilization and reaction configurations not only in batch, but also in continuous processes.

Therefore, this invention refers to a free fatty acids esterification process for the production of biodiesel using niobic acid as a solid acid catalyst. Through the use of the niobic acid it is possible to work with higher conversions, where the water produced in the reaction is higher, with no significant activity loss, and with a higher stability by the minimization of polymerization process of unsaturated fatty acids present in the reaction medium.

### SUMMARY OF INVENTION

This invention consists in a process for the production of biodiesel from the esterification of free fatty acids using niobic acid as heterogeneous catalyst. Biodiesel is a mixture of fatty acids alkyl esters produced by the esterification of free fatty acids with alcohol, performing the reaction in a chemical reactor containing niobic acid as a solid acid catalyst. The free fatty acids result from the refining of vegetable oils and rejects or disposals of industrial, edible, frying, animal fat and organic sewage oils containing significant free acidity. Among the free fatty acids, long chain carboxylic acids containing from 6 to 24 carbon atoms are used in this invention. Short chain alcohols such as methanol and ethanol act as esterifying agent. The niobic acid or the hydrated niobium pentoxide (Nb₂O₅.nH₂O) is used as a solid acid catalyst, but before its use in the reaction medium it goes through a pre-treatment of calcination in order to maximize the acid strength of its catalytic sites. Powdered niobic acid or conformed in tablets or pellets and extrudates are used in this invention. The esterification reaction is conducted in a chemical reactor in batch or continuous mode, where operating variables such as temperature, pressure, type of alcohol, fatty acid/alcohol molar ratio and amount of catalyst are appropriate for the production of biodiesel.

### BRIEF DESCRIPTION OF FIGURES

FIG. 1—Kinetics curve of reaction time versus yield or conversion for palm oil free fatty acids esterification with methanol at the temperature of 130° C., using powdered niobic acid (Nióbia HY®) as a solid acid catalyst and a commercial zeolite Y. Amount of catalyst: (a) 1% in weight; and (b) 2% in weight related to free fatty acids weight.

FIG. 2—Kinetics curve of reaction time versus yield or conversion for palm oil free fatty acids esterification with methanol at the temperature of 130° C., using niobic acid (Nióbia HY®) as tablet or pellet and a commercial zeolite Y. The amount of catalyst was of 2% in weight related to the free fatty acids weight.

FIG. 3—Kinetics curve of reaction time versus yield or conversion for palm oil free fatty acids esterification with methanol at the temperature of 130° C., using extrudates niobic acid (Nióbia HY®). The amount of catalyst was of 2% in weight related to free fatty acids weight.

FIG. 4—Kinetics curve of reaction time versus yield or conversion for palm oil free fatty acids esterification with methanol at the temperature of 130° C., comparing the performance of different forms of niobic acid (Nióbia HY®) and commercial zeolites. The amount of catalyst was of 2% in weight related to the free fatty acids weight.

### DETAILED DESCRIPTION OF INVENTION

The esterification reaction of free fatty acids with an alcohol in the presence of a solid acid catalyst may be represented by the following chemical equation:

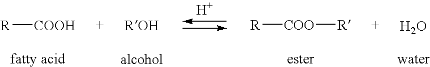

where R represents the fatty acid carbon chain, R′ the alkyl radical of the alcohol used and H⁺ the proton characteristic of the acidity present in the catalyst surface. It must be noted that in addition to the fatty acid alkyl ester, water is one of the reaction products.

Different sources of free fatty acids may be used as raw material. In this invention, free fatty acids result from, but not limited only to these sources, the refining of vegetable oils such as soy, rape, palm, canola, olive, babaçu, peanut, coconut, castor, cotton and sunflower oils. Free fatty acids may also be originated from the disposal of vegetable oils used in frying, such as soy, canola and palm, rejects of industrial oils, animal fat and fat enriched organic sewage where the free acidity is significant.

The composition of free fatty acids used in this invention comprise long chain carboxylic acids containing from 6 to 24 atoms of carbon (C₆ to C₂₄) such as caproic, caprylic, capric, lauric, miristic, palmitic, palmitoleic, stearic, oleic, vaccenic, linoleic, linolenic, arachidic, gadoleic, arachidonic, behenic, erucic and linoceric acids. The presence and proportion of each one of these fatty acids depend on their origin, varying according to the source of fatty acids used. For instance, the free fatty acids from palm oil contain 0.5% of lauric acid, 0.5% of miristic acid, 45% of palmitic acid, 4% of stearic acid, 40% of oleic acid and 10% of linoleic acid. For their turn, the free fatty acids from sunflower oil contain 7% of palmitic acid, 5% of stearic acid, 19% of oleic acid, 68% of linoleic acid and 1% of linolenic acid.

Different types of alcohols may be used as esterifying agent such as methanol, ethanol, propanol, isopropanol, butanol and isobutanol. In this invention, the short chain alcohols such as methanol (CH₃OH) and ethanol (CH₃CH₂OH) are preferred. When methanol is used, the biodiesel obtained is a mixture of fatty acids methyl esters. For its turn, the use of ethanol results in the formation of a mixture of fatty acids ethylic esters. The alcohol is added in excess to the reaction mixture to increase the biodiesel yield, the molar ratio of alcohol and fatty acids preferably varying from 1.5 to 6 in this invention. The amount of catalyst is determined depending on the amount of fatty acids, being contents of 1 to 2% in catalyst weight related to the weight of the fatty acids used in this invention.

The hydrated niobium pentoxide (Nb₂O₅.nH₂O), also known as niobic acid, is the solid acid catalyst used in this invention. The presence of water in its composition is essential for its acidity and control of optimum amount of water through the calcination conditions maximizes the acid strength of its catalytic sites. The optimum contents of water range from 5 to 25% in weight when niobic acid is submitted to the calcination temperature between 100 to 300° C., for periods of 1 to 3 hours. The niobic acid may be used as powder or conformed as tablets or pellets and extrudates. Traditional and known processes in the art for tableting or pelleting and extrusion of catalysts may be used for the conformation of the powdered niobic acid. Tablets or pellets and extrudates are the preferred configurations for the minimization of pressure drop in the chemical reactor, provided that they present a good mechanical resistance and are easy to handle. Mechanical resistance in the range of 10 to 40 N·mm⁻¹ is the preferred values for this invention. Independent of its form, whether powder, tablet/pellet or extrudates, the BET surface area of niobic acid must be within 10 to 300 m²/g, preferably between 80 and 200 m²/g.

The reaction must be conducted under the lowest temperatures possible, in order to increase the lifetime of the catalyst by minimizing the deactivation process cause by fatty acid degradation and formation of coke. The reaction temperature used in this invention is within the range of 80 to 200° C., preferably between 120 and 170° C. Although the increase of pressure in the reaction medium may be beneficial to the reaction process, displacing the equilibrium to the formation of products, the reaction pressure used in this invention is endogenous, resulting from the presence of volatile alcohols such as methanol or ethanol. At a certain reaction temperature the alcohol would create vapors, causing the increase of the reaction pressure. In reaction conditions using methanol at the temperature of 130° C. the endogenous reaction pressure may reach 552 kPa (80 psi).

The reaction configuration of this invention accepts both batch and continuous processes. In choosing batch chemical reactors, with mechanical stirring, the time of residence may range from 60 to 300 minutes of reaction. In choosing continuous reactors in fixed bed or of perfect mixture (CSTR: continuously stirred-tank reactor) the spatial time related to fatty acid may range from 15 to 160 minutes, preferably between 30 and 120 minutes.

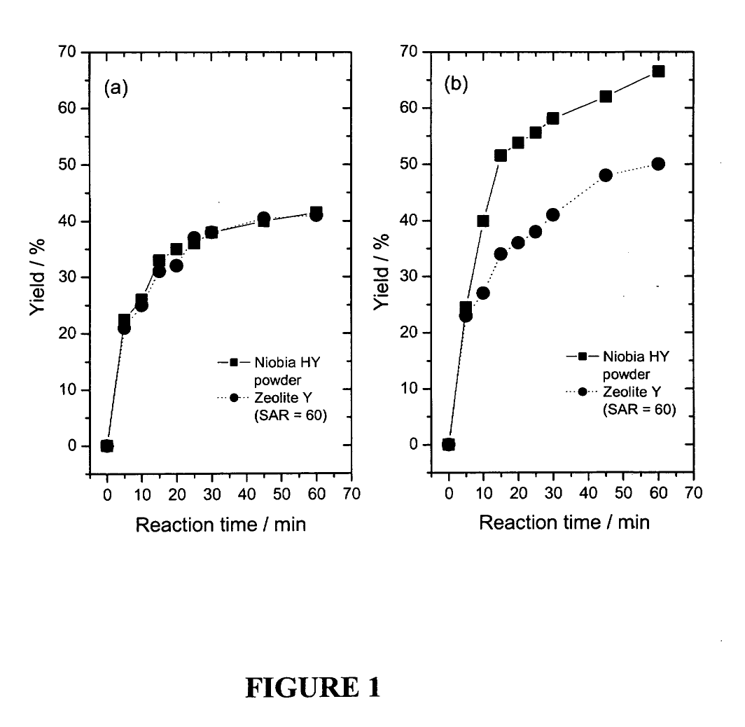

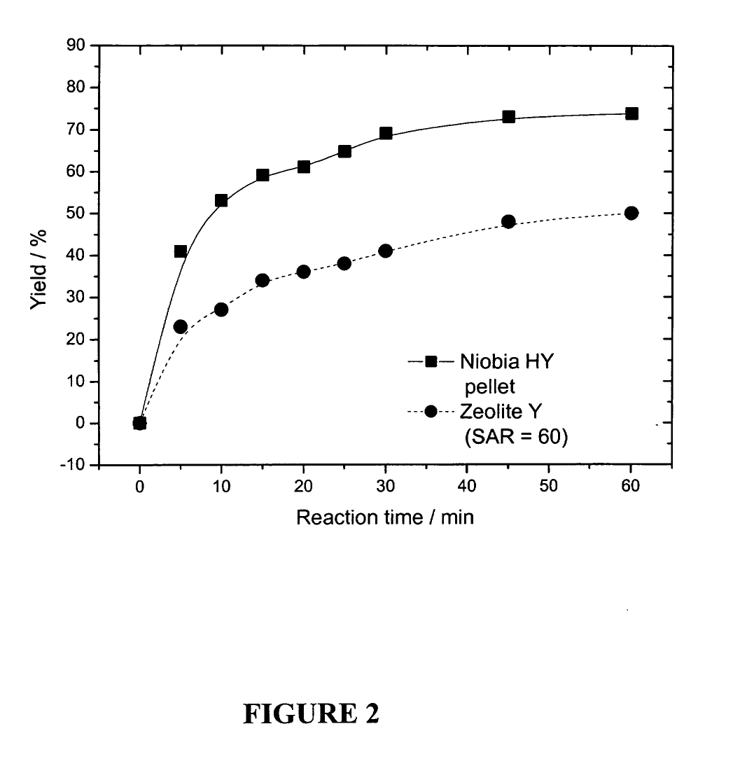

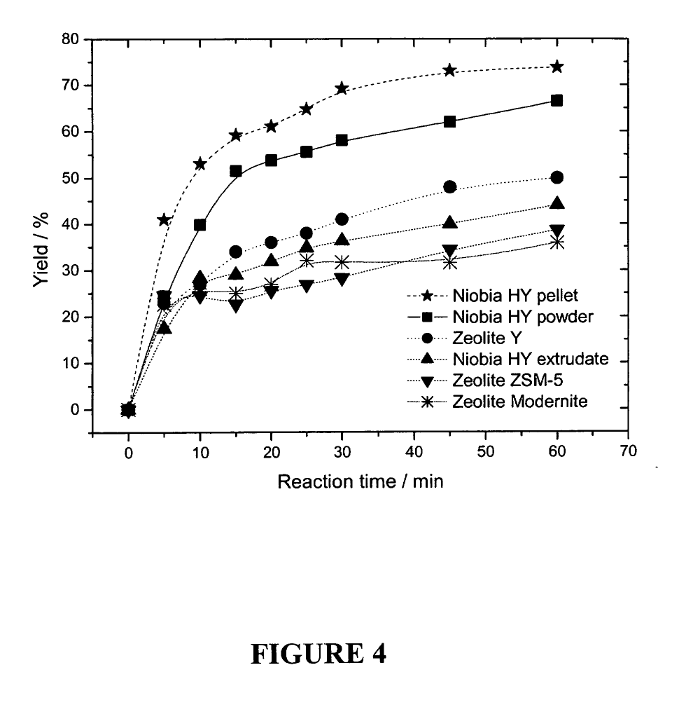

The results obtained from this invention are shown on FIGS. 1 to 4. FIG. 1 shows the effect of the amount of catalyst in the performance of the powdered niobic acid, compared to a commercial zeolite Y (CBV-760 by Zeolyst Inc.) with silica/alumina ratio of 60 (SAR=60), constituting of a very acidic zeolite. For 1% in weight of catalyst (FIG. 1a) the significant difference between niobic acid and zeolite Y is not seen. With the increase of catalyst content (2% in weight, FIG. 1b), the niobic acid presented a better performance after 5 minutes of reaction. This behavior is associated to the ability of the niobic acid in dealing with higher water contents in the reaction medium. In a lower amount of catalyst the water influence is not seen, as the yielding is low, below 50%. With the increase of catalyst content, the yield notably increases and, consequently, more water is formed by the reaction. However, the zeolite Y does not keep the same level of activity of the niobic acid, showing the superiority of the latter in higher water concentrations. The conformation of the niobic acid in tablet/pellet keeps and improves the activity in relation to the powder, being much superior to zeolite Y (FIG. 2). FIG. 4 shows that the niobic acid is much superior not only to zeolite Y but also in relation to other acid zeolites such as ZSM-5 and modemite.

Some examples of the efficacy of the invention described herein are shown below:

### EXAMPLE 1

Use of powdered niobic acid at reaction temperature of 130° C. A mixture of free fatty acids from refined palm oil consisting of 1.2% lauric acid, 0.1% miristic acid, 45.4% palmitic acid, 40.5% oleic acid and 11.1% of linoleic acid and methanol at a molar methanol/fatty acids ratio of 3 to 1 was used as reagent. A sample of powdered niobic acid (Nióbia HY® by CBMM—Cia. Brasileira de Metalurgia e Mineração) was calcined at 300° C. for two hours in aerated muffle, then adding the reaction mixture in an amount corresponding to 1 or 2% in weight of present fatty acids. After calcination, the powdered niobic acid presented a BET surface area of 170 m²/g. The reaction was conducted at 130° C. in 500 mL Parr reactor with mechanical stirring of 500 rpm for a period of 60 min. Reaction samples were collected every 5 minutes to follow-up the reaction kinetics. FIG. 1 shows the results, kinetics curve of time of reaction versus the yield or conversion, for 1% in weight of catalyst (FIG. 1a) and for 2% in weight of catalyst (FIG. 1b).

### EXAMPLE 2

Use of niobic acid in tablet or pellet at reaction temperature of 130° C. The same mixture of free fatty acids and methanol and the same reaction conditions of Example 1 are used in this example. Instead of powdered niobic acid, niobic acid (Nióbia HY® by CBMM—Cia. Brasileira de Metalurgia e Mineração) as tablets or pellets was used, with a mean diameter of around 6 mm and mechanical resistance of 10 N·mm⁻¹. The pellet or tablet was submitted to calcination at a temperature of 300° C. for a period of 1 hour and showed a BET surface area of 150 m²/g. The kinetics curve of time of reaction versus yield or conversion is shown in FIG. 2 to 2% in weight of catalyst.

### EXAMPLE 3

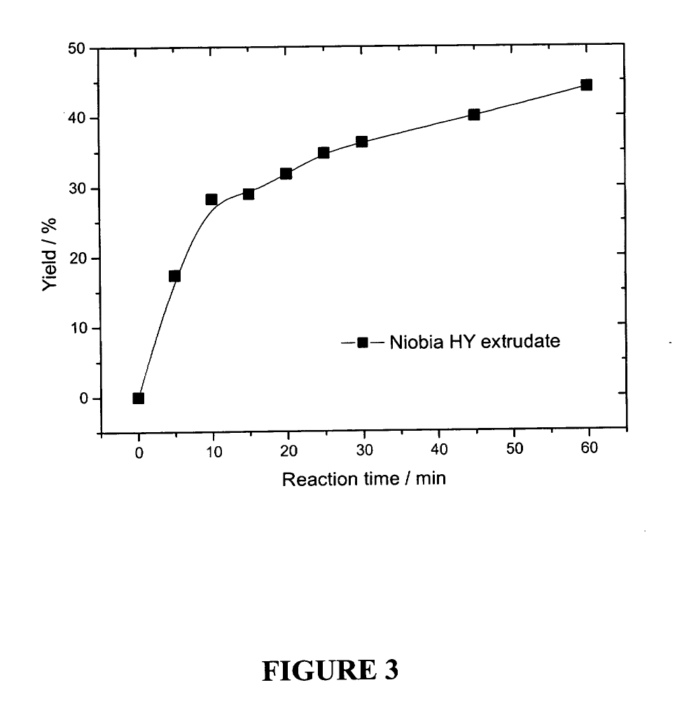

Use of extrudates niobic acid at reaction temperature of 130° C. The same mixture of free fatty acids and methanol and the same reaction conditions of Example 1 are used in this example. Instead of powdered niobic acid, extrudates niobic acid was used (Nióbia HY® by CBMM—Cia. Brasileira de Metalurgia e Mineração), with mean diameter between 2 to 6 mm and mechanical resistance of 30 N·mm⁻¹. The extrudates was submitted to calcination at a temperature of 300° C. for a period of 1 hour and showed a BET surface are of 200 m²/g. The kinetics curve of time of reaction versus yield or conversion is shown in FIG. 3 to 2% in weight of catalyst.

### EXAMPLE 4

Use of niobic acid in tablet or pellet at reaction temperature of 150° C. and 170° C. Mixture of fatty acids and methanol of Example 1. In this example, niobic acid in tablet or pellet was used (Nióbia HY® by CBMM—Cia. Brasileira de Metalurgia e Mineração) that after calcination at 300° C. for 1 hour showed BET surface area of 100 m²/g and mechanical resistance of 40 N·mm⁻¹. The temperatures of reaction were 150 and 170° C. and the results are presented in the table below for 4 sequential runs of 60 minutes, with 2% in weight of catalyst, in 500 mL Parr reactor with mechanical stirring at 500 rpm.

TABLE
Percentage (%) yield after 4 sequential batches of catalyst

| RUN # | 150° C. | 170° C. |
|---|---|---|
| 1 | 53.8 | 89.2 |
| 2 | 51.3 | 75.4 |
| 3 | 47.2 | 60.2 |
| 4 | — | 63.0 |

The results in the table above show that the yielding above 50% are kept even after 4 sequential uses of niobic acid at temperature of 170° C., suggesting that the polymerization processes lead to the graduate deactivation of the catalyst, but not significantly.

### EXAMPLE 5

Comparative use of commercial zeolites: Y with silica/alumina ratio of 60 (SAR=60), ZSM-5 and Modernite, at a reaction temperature of 130° C. A commercial zeolite Y (CBV-760 by Zeolyst Inc.), with silica/alumina ratio of 60, a commercial zeolite ZSM-5 and a commercial modernite zeolite were used as catalysts for the comparison. Zeolites Y, ZSM-5 and Modernite showed BET surface areas above 500 m²/g and were added, 2% in weight and with no previous calcination, in the mixture of fatty acids and methanol of Example 1. The reaction temperature was of 130° C. for a period of 60 minutes. FIGS. 1, 2 and 4 show its performance in comparison to niobic acid in powder, tablet/pellet and extrudates. It is observed that the performance of the niobic acid in powder and tablet/pellet was superior to that of zeolites.

1. A process for the production of Biodiesel characterized by the use of a solid niobic acid catalyst (Nb₂O₅.nH₂O or hydrated niobium pentoxide) in the free fatty acids esterification with an alcohol.

2. A process for the production of Biodiesel according to claim 1, characterized in that the free fatty acids are from refining of vegetable oils such as soy, rape, palm, canola, olive, babaçu, peanut, coconut, castor, cotton and sunflower oils.

3. A process for the production of Biodiesel according to claim 1, characterized in that the free fatty acids are from disposals where free acidity is significant, such as used vegetable oils, rejects of industrial oils, animal fat and fat-enriched organic sewage.

4. A process for the production of Biodiesel according to claims 2 and 3, characterized in that the free fatty acids are long chain carboxylic acids containing 6 to 24 carbon atoms (C₆ to C₂₄) such as caproic, caprylic, capric, lauric, miristic, palmitic, palmitoleic, stearic, oleic, vaccenic, linoleic, linolenic, arachidic, gadoleic, arachidonic, behenic, erucic, linoceric acids or their mixtures.

5. A process for the production of Biodiesel according to claim 1, characterized in that the alcohol is methanol, ethanol, propanol, isopropanol, butanol or isobutanol.

6. A process for the production of Biodiesel according to claim 5, characterized in that the alcohol is preferably methanol and ethanol.

7. A process for the production of Biodiesel according to claim 1, characterized in that the alcohol is added in excess to the reaction mixture, with molar alcohol fatty acids ratio between 1,5 and 6.

8. A process for the production of Biodiesel according to claim 1, characterized in that the water content of niobic acid is within 5 to 25% in weight.

9. A process for the production of Biodiesel according to claim 1, characterized in that the niobic acid is calcined at a temperature of 100 to 300° C.

10. A process for the production of Biodiesel according to claim 9, characterized in that the niobic acid calcination is for a period of 1 to 3 hours.

11. A process for the production of Biodiesel according to claim 1, characterized in that the BET surface area of niobic acid is between 10 to 300 m²/g.

12. A process for the production of Biodiesel according to claim 11, characterized in that the preferred BET surface area is within 80 to 200 m²/g.

13. A process for the production of Biodiesel according to claim 1, characterized in that the niobic acid is used in powder, tablet or pellet and extrudates.

14. A process for the production of Biodiesel according to claim 13, characterized in that that the mechanical resistance of tablet/pellet and extrudates of niobic acid is within 10 to 40 N·mm⁻¹.

15. A process for the production of Biodiesel according to claim 1, characterized in that the amount of niobic acid used in the reaction ranges from 1 to 2% in weight of the mixture of free fatty acids.

16. A process for the production of Biodiesel according to claim 1, characterized in that the reaction temperature is between 80 to 200° C.

17. A process for the production of Biodiesel according to claim 16, characterized in that that the preferred reaction temperature is between 120 to 170° C.

18. A process for the production of Biodiesel according to claim 1, characterized in that the pressure of reaction is endogenous and depends on the temperature and alcohol used.

19. A process for the production of Biodiesel according to claim 1, characterized in that the reaction configuration uses batch reactors.

20. A process for the production of Biodiesel according to claim 1, characterized in that the reaction configuration uses continuous reactors of fixed bed and perfect mixture.

21. A process for the production of Biodiesel according to claim 19, characterized in that the time of residence in the batch reactors ranges between 60 to 300 minutes of reaction.

22. A process for the production of Biodiesel according to claim 20, characterized in that the spatial time in the continuous reactions in relation to the fatty acids flow rate and volume of solid catalyst is between 15 and 160 minutes.

23. A process for the production of Biodiesel according to claim 22, characterized in that the preferred spatial time in continuous reactors in relation to fatty acids flow rate and the volume of solid catalyst is between 30 to 120 minutes.

24. A process for the production of Biodiesel according to claim 1, characterized in that the obtained Biodiesel is a mixture of fatty acids methyl esters.

25. A process for the production of Biodiesel according to claim 1, characterized in that the obtained Biodiesel is a mixture of fatty acids ethyl esters.
